(19) United States
(12) Patent Application Publication
(10) Pub. No.: US 2016/0316790 A1
(43) Pub. Date: Nov. 3, 2016

(54) BETA-HINOKITIOL DERIVATIVES AND APPLICATIONS THEREOF IN PREPARATION OF ANIMAL ANTIBACTERIAL AGENTS AND ANTIBACTERIAL GROWTH PROMOTERS USED IN FEED

(71) Applicant: GUANGZHOU INSIGHTER BIOTECHNOLOGY CO., LTD., Guangzhou City, Guangdong (CN)

(72) Inventors: Xianfeng Peng, Guangzhou (CN); Zonghua Qin, Guangzhou (CN)

(73) Assignee: GUANGZHOU INSIGHTER BIOTECHNOLOGY CO., LTD., Luogang, Guangzhou, Guangdong (CN)

(21) Appl. No.: 15/108,726

(22) PCT Filed: Jan. 14, 2014
(86) PCT No.: PCT/CN2014/070603

§ 371 Date: Jun. 28, 2016

(30) Foreign Application Priority Data
Dec. 31, 2013 (CN) 201310751901.X

(51) Int. Cl. A23K 20/195; A23K 20/111; C07C 49/723; A23K 50/75; C07C 49/717; A01N 35/04

(52) U.S. Cl. CPC A23K 20/195; C07C 49/717; A01N 35/04; C07C 49/723; A23K 50/75; A23K 20/111

(57) ABSTRACT

β-hinokitiol derivatives and application thereof in preparation of animal antibacterial agents and antibacterial growth promoters used in feed. β-hinokitiol derivatives, having a structural formula as shown in formula (I): wherein, R is n-propyl, n-pentyl, benzyl, sec-butyl, ethyl, n-butyl, p-chlorobenzyl, p-fluorobenzyl, n-octyl, 1-fluoropropyl or n-tetradecyl. The compounds as shown in formula I show low toxicity or non-toxicity to poultry, are safer and have strong growth promotion properties, and therefore are suitable to be used as growth promoters in feed. The compounds have a very good application prospect in cultivation industry.

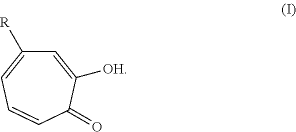

### FIELD OF THE INVENTION

The present invention relates to the field of poultry cultivation, specifically to β-hinokitiol derivatives and application thereof in preparation of an animal antibacterial agent and an antibacterial growth promoter used in feed.

### DESCRIPTION OF THE PRIOR ART

β-hinokitiol, chemical name: 2-hydroxy-4-isopropyl-1-cycloheptatrienone, is a natural edible essential oil ingredient, and has been used as a food additive for years in Japan.

U.S. Pat. No. 6,387,417B1 discloses activities of β-hinokitiol and complexes (2:1 or 3:1) inhibiting Enterococcus resistant to Vancomycin, and application thereof in treatment.

### SUMMARY OF THE INVENTION

The first object of the present invention is to provide β-hinokitiol derivatives which have broad-spectrum antibacterial activities and can be used as animal antibacterial agents and antibacterial growth promoters used in feed.

β-hinokitiol derivatives of the present invention have a structural formula as shown in formula (I).

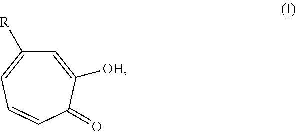

Wherein, R is n-propyl, n-pentyl, benzyl, sec-butyl, ethyl, n-butyl, p-chlorobenzyl, p-fluorobenzyl, n-octyl, 1-fluoropropyl or n-tetradecyl.

The specific structural formula is shown as below:

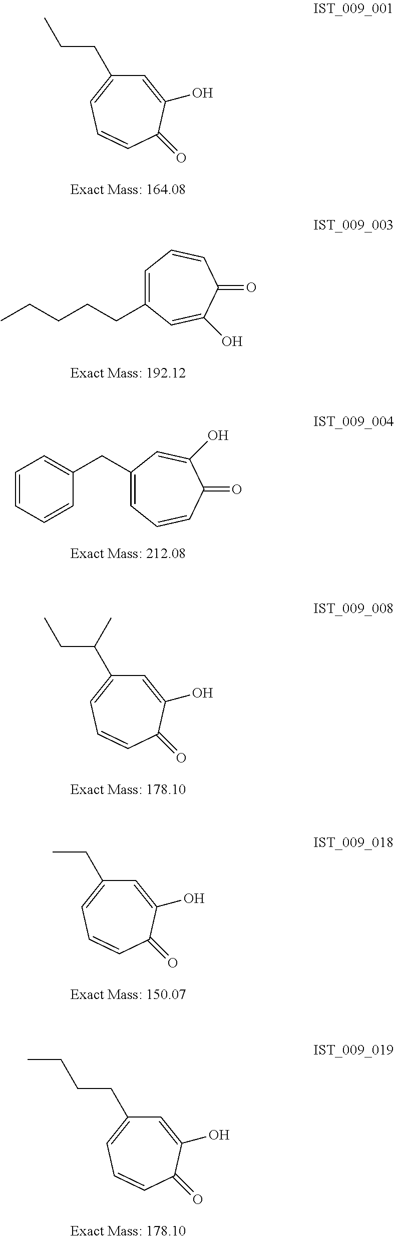 [SMILES: CCCc1cccc(=O)c(O)c1 ; CCCCCc1cccc(=O)c(O)c1 ; O=c1cccc(Cc2ccccc2)cc1O ; CCC(C)c1cccc(=O)c(O)c1 ; CCc1cccc(=O)c(O)c1 ; CCCCc1cccc(=O)c(O)c1]

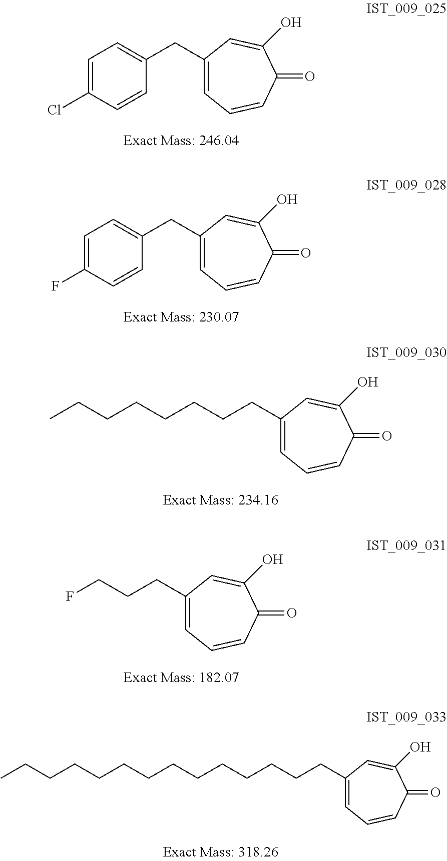 [SMILES: O=c1cccc(Cc2ccc(Cl)cc2)cc1O ; O=c1cccc(Cc2ccc(F)cc2)cc1O ; CCCCCCCCc1cccc(=O)c(O)c1 ; O=c1cccc(CCCF)cc1O ; CCCCCCCCCCCCCCc1cccc(=O)c(O)c1]

Through experiments, it has been found that the β-hinokitiol derivatives of the present invention have broad-spectrum antibacterial activities, show high inhibitory activities on major animal pathogenic bacteria (for example, clostridium perfringens, staphylococcus aureus, escherichia coli, salmonella, riemerella anatipestifer, haemophilus parasuis, vibrio and streptococcus), and in particular that, the antibacterial activities of additives such as IST_009_001 and IST_009_003 are better than those of the lead compound IST_001_002 (β-hinokitiol). Besides, through broiler feeding experiments, it has been found that β-hinokitiol derivatives such as IST_009_001 and IST_009_003 can enhance the weight gain and the feed conversion efficiency of the experimental broilers. Through weaned pig feeding experiments, it has been found that adding IST_009_001 in feeds can obviously reduce the diarrhea rate of the experimental pigs, and the effect of IST_009_001 is better than that of IST_001_002.

Therefore, the second object of the present invention is to provide applications of any one of the above-mentioned β-hinokitiol derivatives in preparation of animal antibacterial agents and antibacterial growth promoters used in feed.

The animals include pigs, chickens, ducks, geese, beef cattle, dairy cattle, sheep, fish, shrimp, foxes, martens or raccoon dogs in all growth stages.

Dosage of the β-hinokitiol derivative in animal feeds is 0.1˜200 ppm.

When the animal antibacterial agents are used to treat diseases caused by infection with various animal sensitive bacteria, the dosage is 1-30 mg/kg (body weight).

The compounds of the present invention as shown in formula I show low toxicity or non-toxicity to poultry, is safer and has stronger growth promotion properties in comparison with all patented compounds at present, and therefore is more suitable to be used as a growth promoters used in feed. The present invention has a very good application prospect in cultivation industry.

### DETAILED DESCRIPTION OF THE EMBODIMENTS

The present invention is described in further detail with reference to embodiments which shall not be regarded as limits to the present invention.

11 β-hinokitiol derivatives of the present invention have structural formulas as shown below:

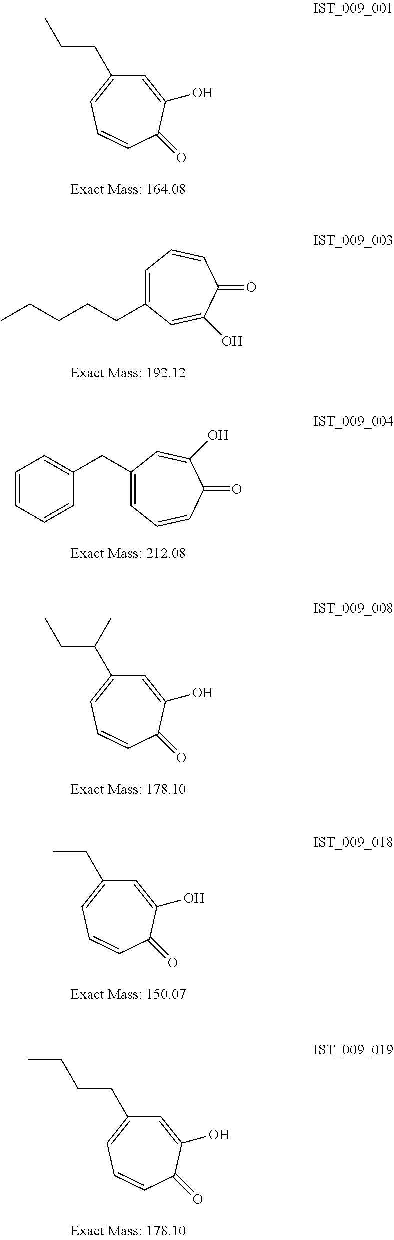 [SMILES: CCCc1cccc(=O)c(O)c1 ; CCCCCc1cccc(=O)c(O)c1 ; O=c1cccc(Cc2ccccc2)cc1O ; CCC(C)c1cccc(=O)c(O)c1 ; CCc1cccc(=O)c(O)c1 ; CCCCc1cccc(=O)c(O)c1]

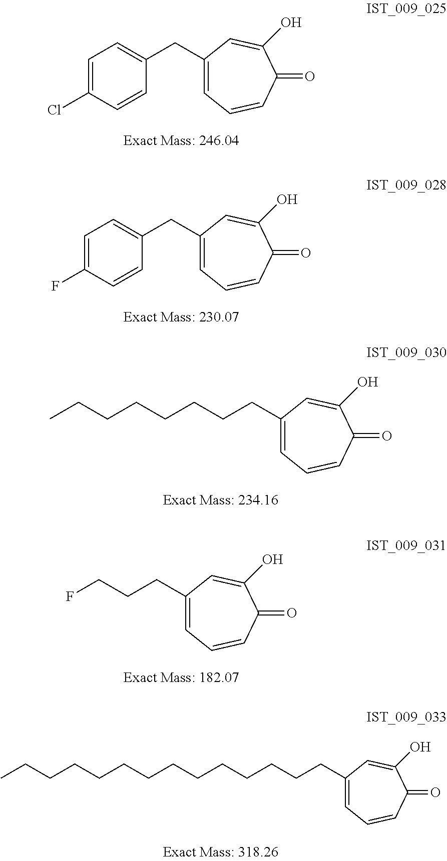 [SMILES: O=c1cccc(Cc2ccc(Cl)cc2)cc1O ; O=c1cccc(Cc2ccc(F)cc2)cc1O ; CCCCCCCCc1cccc(=O)c(O)c1 ; O=c1cccc(CCCF)cc1O ; CCCCCCCCCCCCCCc1cccc(=O)c(O)c1]

### Embodiment 1

### Synthesis of IST_009_001 (2-hydroxy-4-propylcyclohepta-2,4,6-trienone)

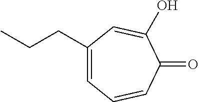

### Preparation of 2-hydroxy-4-propylcyclohepta-2,4,6-trienone

Step 1:

At room temperature, to a 500 mL three-necked flask was added 150 ml of DMSO, stirred vigorously and purged with N₂ for 30 min. Potassium hydroxide (0.3 mol, 1 eq) was added and the mixture was continuously stirred for 0.5 h. Then cyclopentadiene monomers (0.8˜1.2 eq) was added dropwise within 30 min and the resulting mixture was continuously stirred for 1 h under N₂. Then, the mixture is cooled to about −10° C.; the mixture of n-hexane (150 ml) and bromopropane (0.8˜1.2 eq) was added dropwise into the three-necked flask for about 1 h, with the temperature controlled to be not higher than 20° C. And then the reaction continued for 1 h at room temperature; then, diluted hydrochloric acid was added dropwise to adjust the pH to be acidic; after the adjustment was completed, the mixture was stirred for 5-10 min and then separated; the water layer was extracted one more time with 150 ml of n-hexane; the combined organic layers was washed with 250 ml of water twice until the water phase becomes neutral, dried over anhydrous sodium sulfate 40 g, and filtrated, and then the filtrate (about 400 ml) was added into a 1,000 ml three-necked flask and let standing for 7-20 h at a temperature of 20±2° C. to achieve isomerization wherein gas chromatography (GC) was performed to determine the degree of isomerization, and n-propyl cyclopentadiene was obtained when the result is good.

Step 2:

Under stirring, dichloroacetyl chloride (0.7˜1.5 eq) was added into the n-hexane solution of the n-propyl cyclopentadiene; after the mixture was cooled to about −15˜10° C., triethylamine (0.8˜1.5 eq) was added dropwise within 1 h at the temperature controlled to be not higher than 15° C., and then the reaction proceeded for 1 h at a constant temperature; then, diluted hydrochloric acid was added dropwise to adjust the pH to be neutral or subacid; the solution was stirred for 5-10 min and then separated, wherein concentration of the product in each of the resulting organic phase and the water phase was determined by thin layer chromatography (TLC, and with PE as a developing solvent). The organic phase was then subjected to rotary evaporation at 35° C. to remove solvent, and the residual green liquid was dichlorobicycloketone.

Step 3:

The dichlorobicycloketone prepared in the previous step, 125 ml of tertiary butanol, 20 ml of acetic acid and 25 ml of water were added in sequence into a 1,000 ml three-necked flask with a condenser pipe. The resulting reaction mixture was stirred at 75˜80° C.; 85 ml (98%, 0.675 mol) triethylamine was added dropwise (for about 1 h), and then the mixture was kept in a constant temperature overnight. The reaction was monitored by TLC with petroleum ether (PE) as a developing solvent. At the completion of the reaction, the mixture was cooled to a temperature below 40° C., 300 ml of dichloromethane was added into the mixture resulting in precipitation of a great amount of solids, and then diluted hydrochloric acid was added dropwise to adjust the pH to be acidic. The resulting mixture was stirred for 5-10 min and then the organic phase was moved out. The precipitate in the flask was washed with 100 ml of dichloromethane, and then the organic phase was moved out. Aqueous solution of potassium hydroxide (3M) was added to the combined organic phases, wherein the product is dissolved in the water phase, and a TLC test of the dichloromethane layer showed impurity spots but no product spot. Extraction and liquid separation were performed, the organic layer was removed, and the water phase was washed again with petroleum ether. The water phase was then added with petroleum ether, and then the mixed solution was added with aqueous solution of hydrochloric acid (3M) with stirring until the solution turns into acidic; the water phase was then removed, and the organic phase was washed with water three times. TLC of the organic phase (PE:EA=5:1) showed one spot. NMR result showed that the product is 2-hydroxy-4-propylcyclohepta-2,4,6-trienone, a structural formula of which is:

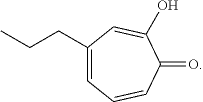

2-hydroxy-4-propylcyclohepta-2,4,6-trienone: δH (DMSO, 500 MHz) 7.271-7.313 (1H, m), 7.144 (1H, s), 7.073-7.095 (1H, d, J=11), 6.912-8.932 (1H, d, J=10), 2.530-2.560 (2H, m), 1.544-1.618 (2H, m), 0.867-0.896 (3H, m).

### Embodiment 2

### Synthesis of IST_009_003 (2-hydroxy-4-pentylcyclohepta-2,4,8-trienone)

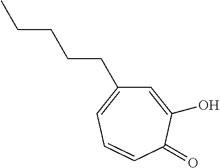

### Preparation of 2-hydroxy-4-pen cyclohepta-2,4,6-trienone

Step 1:

At room temperature, to a 500 mL three-necked flask was added 150 ml of DMSO, stirred vigorously and purged with N₂ for 30 min. Potassium hydroxide (0.3 mol, 1 eq) was added and the mixture was continuously stirred for 0.5 h. Then cyclopentadiene monomers (0.8˜1.2 eq) was added dropwise within 30 min, and the mixture was continuously stirred for 1 h. Then, the mixture was cooled to about −10° C.; a mixture of n-hexane (150 ml) and 1-bromopentane (0.8˜1.2 eq) was added dropwise into the three-necked flask within 1 h, at the temperature controlled to be not higher than 20° C. And then the reaction continued for 1 h or more in a self-heating process; then, diluted hydrochloric acid was added dropwise to adjust the pH to be acidic; after the adjustment was completed, the mixture was stirred for 5-10 min and then separated; the water layer was extracted one more time with 150 ml of n-hexane; the combined organic layers was washed by 250 ml of water twice until the water phase becomes neutral, dried over 40 g of anhydrous sodium sulfate, and filtrated, and then the filtrate (about 400 ml) was added into a 1,000 ml three-necked flask and let standing for 7-20 h at a temperature of 20±2° C. to achieve isomerization wherein a GC was performed to determine the degree of isomerization, and n-pentyl cyclopentadiene was obtained.

Step 2:

Under stirring, dichloroacetyl chloride (0.7˜1.5 eq) was added into the n-hexane solution of the n-pentyl cyclopentadiene; after the mixture was cooled to about −15˜10° C., triethylamine (0.8˜1.5 eq) was added dropwise for about 1 hr with the temperature controlled to be not greater than 15° C., and then the reaction proceeded for 1 h at a constant temperature; then, diluted hydrochloric acid was added dropwise to adjust the pH to be neutral or subacid; the solution was stirred for 5-10 min and then separated, wherein concentration of the product in each of the resulting organic phase and the water phase was determined by TLC (with PE as a developing solvent). The organic phase was then subjected to rotary evaporation at a temperature of 35° C. to remove solvent, and the residual green liquid was dichlorobicycloketone.

Step 3:

The dichlorobicycloketone prepared in the previous step, 125 ml of tertiary butanol, 20 ml of acetic acid and 25 ml of water were added in sequence into a 1,000 ml three necked flask with a condenser pipe, stirred and heated to a temperature of 75˜80° C.; 85 ml (98%, 0.675 mol) triethylamine was added dropwise (for about 1 h), and then the mixture was kept in a constant temperature overnight for reaction, and The reaction was monitored by TLC with petroleum ether (PE) as a developing solvent. At the completion of the reaction, the mixture was cooled to a temperature below 40° C., 300 ml of dichloromethane was added into the mixture resulting in precipitation of a great amount of solids, and then diluted hydrochloric acid was added dropwise to adjust the pH to be acidic. Then the solution was stirred for 5-10 min and then separated, wherein TLC (developing solvent: EA:PA=1:5) result showed no presence of product in the resulting water layer. The organic phase (the lower layer) was dried over 40 g of anhydrous sodium sulfate for 15 min, filtrated and distilled at 35˜38° C. to remove solvent. Residues were heated to 265° C. and subjected to high vacuum distillation (with a rotary vane vacuum pump, above −0.098 MPa); and fractions were collected at a temperature of 112˜116° C. At the completion of the distillation, nitrogen gas was introduced into the system. The fractions (about 8 g) were added into 50 ml petroleum ether under nitrogen protection wherein TLC (PE:EA=5:1) result shows two impurity spots above the production spot. The product was completely dissolved and the solution was placed in a refrigerator at a temperature of 4° C., wherein no solids separate out, and oil appears when it is cooled to −20° C.

The product was dissolved in petroleum ether, and then aqueous solution of potassium hydroxide (3M) was added into the mixture, wherein the product was dissolved in the water phase, and a TLC test of the dichloromethane layer showed impurity spots but no product spot. Extraction and liquid separation were performed, the organic layer was removed, and the water phase was washed again with petroleum ether. The water phase was then added with petroleum ether; and then the mixed solution was added with aqueous solution of hydrochloric acid (3M) with stirring until the solution turned into acidic; the water phase was then removed, and the organic phase was washed with water for three times. TLC of the organic phase (PE:EA=5:1) showed one spot. The organic phase was then subject to rotary evaporation to obtain a product. NMR result showed that the product was 2-hydroxy-4-pentylcyclohepta-2,4,6-trienone, a structural formula of which is:

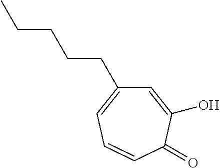

2-hydroxy-4-pentylcyclohepta-2,4,6-trienone: δH (DMSO, 500 MHz) 7269-7.310 (1H, m), 7.142 (1H, s), 7.071-7.093 (1H, d, J=11), 6.912-6.932 (1H, d, J=10), 2.544-2.575 (2H, 1.526-1.584 (2H, m), 1.268-1.292 (4H, m), 0.828-0.854 (3H, m).

### Embodiment 3

### Synthesis of IST_009_004

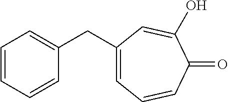 [SMILES: O=c1cccc(Cc2ccccc2)cc1O]

### Preparation of 4-benzyl-2-hydroxycyclohepta-2,4,6-trienone

Step 1:

At room temperature, to a 500 mL three-necked flask was added 150 ml of DMSO, stirred and purged with N₂. Under the N₂ protection, potassium hydroxide (0.3 mol, 1 eq) was added and the mixture was continuously stirred for 0.5 h. Then cyclopentadiene monomers (0.8˜1.2 eq) was added dropwise within 30 min, and the resulting mixture was continuously stirred for 1 h under the N₂ protection. Then, the mixture was cooled to about −10° C., a mixed solution of n-hexane (150 ml) and benzyl bromide (0.8˜1.2 eq) was added dropwise into the three-necked flask for about 1 h, with the temperature controlled to be not higher than 20° C. And then the reaction continued for 1 h or more in a self-heating process; then, diluted hydrochloric acid was added dropwise to adjust the pH to be acidic; after the adjustment was completed, the mixture was fully stirred for 5-10 min and then separated; the water layer was extracted one more time with 150 ml of n-hexane; the combined organic layers were washed with 250 ml of water twice until the water phase was neutral, dried over 40 g of anhydrous sodium sulfate, and filtrated, and then the filtrate (about 400 ml) is added into a 1,000 ml three-necked flask and let standing for 7-20 h at a temperature of 20±2° C. to achieve isomerization wherein a GC was performed to determine the degree of isomerization, and benzyl cyclopentadiene was obtained.

Step 2:

Under stirring, dichloroacetyl chloride (0.7˜1.5 eq) was added into the n-hexane solution of the benzyl cyclopentadiene; the resulting mixture was cooled to about −15˜10° C., triethylamine (0.8˜1.5 eq) was added dropwise for about 1 hr with the temperature controlled to be not greater than 15° C., and then the reaction proceeded for 1 h at a constant temperature; then, diluted hydrochloric acid was added dropwise to adjust the pH to be neutral or subacid; the solution was stirred for 5-10 min and then separated, wherein concentration of the product in each of the resulting organic phase and the water phase was determined by TLC (with PE as a developing solvent). The organic phase was then subjected to rotary evaporation at 35° C. to remove solvent, and the residual green liquid was dichlorobicycloketone.

Step 3:

The dichlorobicycloketone prepared in the previous step, 125 ml of tertiary butanol, 20 ml of acetic acid and 25 ml of water were added in sequence into a 1,000 ml three-necked flask with a condenser pipe and heated to a temperature of 75˜80° C. with stirring; 85 ml (98%, 0.675 mol) triethylamine was added dropwise (for about 1 h), and then the resulting mixture was kept in a constant temperature overnight for reaction. The reaction was monitored by TLC with PE as a developing solvent. At the completion of the reaction, the mixture was cooled to a temperature below 40° C., 150 ml of dichloromethane was added into the mixture, and then diluted hydrochloric acid (10 ml of concentrated hydrochloric acid +200 ml of water) was added dropwise to adjust the pH to be acidic. After the pH adjustment, the solution was stirred for 5-10 min and then separated, wherein TLC (developing solvent: EA:PA=1:5) result showed no presence of product in the resulting water layer. To the organic phase (the lower layer) was added 200 ml of water, followed by 150 ml of aqueous solution of potassium hydroxide (3M) with stirring; and then was subject to extraction without separation. Haft of the upper layer was added into about 200 ml of petroleum ether for extraction and separation. The water layer was washed for one time with petroleum ether. Then, the water layer was added with 200 ml of petroleum ether, and diluted hydrochloric acid (3M) which was added into the mixture with stirring to adjust the pH to 1-2, for extraction and separation into a new water layer and a new organic layer, wherein solids separates out of the organic layer. The organic layer was added with a small amount of ethyl acetate to completely dissolve the solids, and then subject to extraction and separation into a new water layer and a new organic layer. The organic layer was subject to rotary evaporation until about 50 ml thereof remains, placed in a refrigerator overnight to crystallize and filtrated to collect the crystal product. The crystals were washed with petroleum ether and thereafter 2.2 g of product was obtained. NMR result showed that the product is 4-benzyl-2-hydroxycyclohepta-2,4,6-trienone, a structural formula of which is:

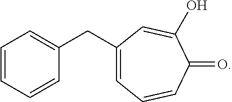 [SMILES: O=c1cccc(Cc2ccccc2)cc1O]

4-benzyl-2-hydroxycyclohepta-2,4,6-trienone: δH (DMSO, 500 MHz) 7.303-7.316 (3H, m), 7.269-7.283 (2H, d, J=7), 7.211-7.239 (1H, m), 7.183 (1H, s), 7.066-7.088 (1H, d, J=11), 6.985-7.005 (1H, d, J=10), 3.952 (2H, s).

### Embodiment 4

### Synthesis of IST_009_008

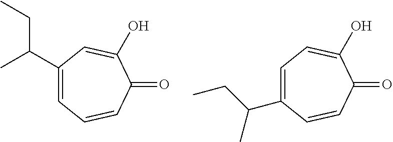

### Preparation of 4-(sec-butyl)-2-hydroxycyclohepta-2,4,6-trienone and 5-(sec-butyl)-2-hydroxycyclohepta-2,4,6-trienone

Step 1:

At room temperature, to a 500 mL three-necked flask was added 150 ml DMSO, stirred and purged with N₂ for 30 min to replace the air in the flask. Under the N₂ protection, potassium hydroxide (0.3 mol, 1 eq) was added and the mixture was continuously stirred for 0.5 h. Then cyclopentadiene monomers (0.8˜1.2 eq) was added dropwise within 30 min, and the resulting mixture was continuously stirred for 1 h under the N₂ protection. Then, the mixture was cooled to about −10° C.; a mixture of n-hexane (150 ml) and 2-bromobutane (0.8˜1.2 eq) was added dropwise into the three-necked flask for about 1 h with the temperature controlled to be not higher than 20° C. And then the reaction continued for 1 h or more in a self-heating process; then, diluted hydrochloric acid was added dropwise to adjust the pH to be acidic and the resulting mixture was stirred for 5-10 min and then separated; the water layer was extracted one more time with 150 ml of n-hexane; the combined organic layers was washed with 250 ml of water twice until the water phase became neutral, dried over 40 g of anhydrous sodium sulfate and filtrated. Then the resulting filtrate (about 400 ml) was added into a 1,000 ml three-necked flask and let standing for 7-20 h at a temperature of 20±2° C. to achieve isomerization wherein a GC was performed to determine the degree of isomerization, and sec-butyl cyclopentadiene was obtained.

Step 2:

Under stirring, dichloroacetyl chloride (0.7˜1.5 eq) was added into the n-hexane solution of the sec-butyl cyclopentadiene; after the mixture was cooled to about −15˜10° C., triethylamine (0.8˜1.5 eq) was added dropwise for about 1 hr with the temperature controlled to be not higher than 151 C, and then the reaction proceeded for 1 h at a constant temperature; then, diluted hydrochloric acid was added dropwise to adjust the pH to be neutral or subacid; the solution was stirred for 5-10 min and then separated, wherein concentration of the product in each of the resulting organic phase and the water phase was determined by TLC (with PE as a developing solvent). The organic phase was then subjected to rotary evaporation at a temperature of 35° C. to remove solvent, and the residual green liquid was dichlorobicycloketone.

Step 3:

The dichlorobicycloketone prepared in the previous step, 125 ml of tertiary butanol, 20 ml of acetic acid and 25 ml of water were added in sequence into a 1,000 ml three-necked flask with a condenser pipe, and the reaction mixture was heated to a temperature of 75˜80° C. with stirring; 85 ml (98%, 0.675 mol) triethylamine was added dropwise (for about 1 h), and then the resulting mixture was kept in a constant temperature overnight, and the reaction was monitored by TLC with PE as a developing solvent. At the completion of the reaction, the mixture was cooled to a temperature below 40° C.; 300 ml of dichloromethane was added into the mixture resulting in precipitation of a great amount of solids. The solids were separated from the liquid, washed with petroleum ether, and then filtrated to obtain another organic phase. The combined organic phase was washed with 200 mL of distilled water and subjected to rotary evaporation to remove the solvent. The residues were subject to decompression distillation and fractions of 100-111° C. were collected with a vacuum degree of −0.099 and a bath temperature of 170-175° C.

The collected fractions were dissolved in 150 ml of petroleum ether in a low-temperature reactor. The mixture was cooled from room temperature to −25° C. so that white crystals was separated out, and then was subjected to filtration. Soon the solids melted, and the resulting oily substances were washed with petroleum ether and thereafter white solid appeared. NMR result showed that the white solid were 5-(sec-butyl)-2-hydroxycyclohepta-2,4,6-trienone. Then the filtrate was subjected to rotary evaporation to remove the solvent, and thereafter 4-(sec-butyl)-2-hydroxycyclohepta-2,4,6-trienone and 5-(sec-butyl)-2-hydroxycyclohepta-2,4,6-trienone were obtained.

4-(sec-butyl)-2-hydroxycyclohepta-2,4,6-trienone: δH (DMSO, 500 MHz) 7.302-7.344 (1H, m), 7.127 (1H, s), 7.085-7.106 (1H, d, J=10.5), 6.097-6.927 (1H, d, J=10), 2.535-2.604 (1H, m), 1.503-1.561 (2H, m), 1.152-1.165 (3H, d, J=6.5), 0.738-0.768 (3H, m)

5-(sec-butyl)-2-hydroxycyclohepta-2,4,6-trienone: δH (DMSO, 500 MHz) 7.268-7.291 (2H, d, J=11.5), 7.167-7.191 (2H, d, J=12), 2.527-2.614 (1H, m), 1.477-1.584 (2H, m), 1.153-1.167 (3H, d, J=7), 0.743-0.773 (3H, m).

### Embodiment 5

### Synthesis of IST_009_018 (4-ethyl-2-hydroxycyclohepta-2,4,6-trienone)

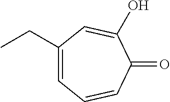

### Preparation of 4-ethyl-2-hydroxycyclohepta-2,4,6-trienone

Step 1:

At room temperature, to a 500 ml three-necked flask was added 150 ml of DMSO, stirred and purged with N₂ for 30 min to replace the air in the flask. Under the N₂ protection, potassium hydroxide (0.3 mol, 1 eq) was added and the mixture was continuously stirred for 0.5 h. Then cyclopentadiene monomers (0.8˜1.2 eq) was added dropwise within 30 min, and the mixture was continuously stirred for 1 h under the N₂ protection. Then, the mixture was cooled to about −10° C.; a mixed solution of n-hexane (150 ml) and bromoethane (0.8˜1.2 eq) was added dropwise into the three-necked flask for about 1 h, with the temperature controlled to be not higher than 20° C. And then the reaction continued for 1 h or more in a self-heating process; then, diluted hydrochloric acid was added dropwise to adjust the pH to be acidic; the resulting mixture was stirred for 5-10 min and then separated; the water layer was extracted with 150 ml of n-hexane; the combined organic layers was washed by 250 ml of water twice until the water phase becomes neutral, dried over 40 g of anhydrous sodium sulfate and filtrated. Then the filtrate (about 400 ml) was added into a 1,000 ml three-necked flask and let standing for 7-20 h at a temperature of 20±2° C. to achieve isomerization wherein a GC was performed to determine the degree of isomerization, and ethyl cyclopentadiene was obtained.

Step 2:

Under stirring, dichloroacetyl chloride (0.7˜1.5 eq) was added into the n-hexane solution of the ethyl cyclopentadiene; after the mixture was cooled to about −15˜10° C., triethylamine (0.8˜1.5 eq) was added dropwise for about 1 hr with the temperature controlled to be not greater than 15° C., and then the reaction proceeded for 1 h at a constant temperature; then, diluted hydrochloric acid was added dropwise to adjust the pH to be neutral or subacid; the resulting solution was stirred for 5-10 min and separated, wherein concentration of the product in each of the resulting organic phase and the water phase was determined by TLC (with PE as a developing solvent). The organic phase was then subjected to rotary evaporation at a temperature of 35° C. to remove solvent, and the residual liquid was dichlorobicycloketone.

Step 3:

The dichlorobicycloketone prepared in the previous step, 125 ml of tertiary butanol, 20 ml of acetic acid and 25 ml of water were added in sequence into a 1,000 ml three-necked flask with a condenser pipe and heated to a temperature of 75˜80° C. with stirring; 85 ml (98%, 0.675 mol) triethylamine was added dropwise (for about 1 h), and then the mixture was kept in a constant temperature overnight. The reaction was monitored by TLC with PE as a developing solvent. The resulting mixture was cooled to a temperature below 40° C. To the mixture were added 150 ml of dichloromethane and diluted hydrochloric acid (10 ml of concentrated hydrochloric acid +200 ml of water) dropwise to adjust the pH to be acidic. After the pH adjustment, the solution was stirred for 5-10 min and then separated, wherein TLC (developing solvent EA:PA=1:5) result showed no presence of product in the resulting water layer. The organic phase was subjected to rotary evaporation to remove the solvent and then added into 200 ml of petroleum ether. The mixture is stirred for half an hour and then let standing for separation into layers wherein the lower layer was black viscous liquid, and the upper layer was dark organic phase. The two layers were separated and the black viscous liquid was discarded. The organic phase (the upper layer) was added with 200 ml of water and then followed by 150 ml of aqueous solution of potassium hydroxide (3M) with stirring for extraction. The water layer was washed with petroleum ether. Then, the water layer was added with 200 ml of petroleum ether, and diluted hydrochloric acid (3M) which was added into the mixture with stirring to adjust the pH to 1-2 for extraction. The organic layer was washed with water and subjected to rotary evaporation to remove the solvent and obtain a product, purity of which was 96% by HPLC. NMR result showed that the product was 4-ethyl-2-hydroxycyclohepta-2,4,6-trienone, a structural formula of which is:

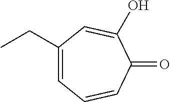

4-ethyl-2-hydroxycyclohepta-2,4,6-trienone: δH (DMSO, 500 MHz) 7.271-7.315 (1H, m), 7.144 (1H, s), 7.072-7.094 (1H, d, J=11), 6.917-6.937 (1H, d, J=10), 2.564-2.609 (2H, m), 1.146-1.176 (3H, m).

### Embodiment 6

### Synthesis of IST_009_019

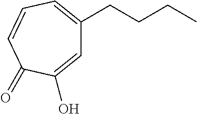

### Preparation of 4-butyl-2-hydroxycyclohepta-2,4,6-trienone

Step 1:

At room temperature, to a 500 ml three-necked flask was added 150 ml of DMSO, stirred and purged with N₂ for 30 min to replace the air in the flask. Under the N₂ protection, potassium hydroxide (0.3 mol, 1 eq) was added and the mixture was continuously stirred for 0.5 h. Then cyclopentadiene monomers (0.8˜1.2 eq) was added dropwise within 30 min. The resulting mixture was continuously stirred for 1 h under the N₂ protection. Then, the mixture was cooled to about −10° C.; a mixture of n-hexane (150 ml) and n-butyl bromide (0.8˜1.2 eq) was added dropwise into the three-necked flask for about 1 h, with the temperature controlled to be not higher than 20° C. And then the reaction continued for 1 h or more in a self-heating process; then, diluted hydrochloric acid was added dropwise to adjust the pH to be acidic and the resulting mixture was fully stirred for 5-10 min and then separated; the water layer was extracted with 150 ml of n-hexane; the combined organic layers was washed with 250 ml of water twice until the water phase became neutral, dried over 40 g of anhydrous sodium sulfate and filtrated. Then the filtrate (about 400 ml) was added into a 1,000 ml three-necked flask and let standing for 7-20 h at a temperature of 20±2° C. to achieve isomerization wherein a GC was performed to determine the degree of isomerization, and n-butyl cyclopentadiene was obtained.

Step 2:

Under stirring, dichloroacetyl chloride (0.7˜1.5 eq) was added into the n-hexane solution of the n-butyl cyclopentadiene; after the mixture was cooled to about −15˜10° C., triethylamine (0.8˜1.5 eq) was added dropwise within 1 h with the temperature controlled to be not greater than 15° C., and then the reaction proceeded for 1 h at a constant temperature; then, diluted hydrochloric acid was added dropwise to adjust the pH to be neutral or subacid; the solution was stirred for 5-10 min and then separated, wherein concentration of the product in each of the resulting organic phase and the water phase was determined by TLC (with PE as a developing solvent). The organic phase was then subjected to rotary evaporation at a temperature of 35° C. to remove solvent, and the residual liquid is dichlorobicycloketone.

Step 3:

The dichlorobicycloketone prepared in the previous step, 125 ml of tertiary butanol, 20 ml of acetic acid and 25 ml of water were added in sequence into a 1,000 ml three-necked flask with a condenser pipe and heated to a temperature of 75˜80° C. with stirring; 85 ml (98%, 0.675 mol) triethylamine was added dropwise within 1 hour, and then the mixture was stirred at a constant temperature overnight, and the reaction was monitored by TLC with PE as a developing solvent. At the completion of the reaction, the mixture was cooled to a temperature below 40° C. To the above mixture were added 200 ml of ethyl acetate, 200 ml of water and 10 ml of concentrated hydrochloric acid for extraction. The organic layer was washed twice with 200 ml of water and subjected to rotary evaporation to remove the solvent. The residue was distilled with a pressure of −0.1 MPa and a external temperature of 150˜180 C. The fraction at 115-120° C. were collected and 25 g of product was obtained. The product is dissolved in the mixture of 150 ml of n-hexane and 10 ml of ethanol. The mixture was heated to 70° C. so that the product is completely dissolved. Then, the mixture was placed in a low-temperature tank cooled to a temperature of −5° C. wherein a great amount of white crystals was observed and collected by filtration. The filter cake was washed with n-hexane to obtain a product. NMR result showed that the product was 4-butyl-2-hydroxycyclohepta-2,4,6-trienone, a structural formula of which is:

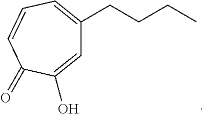

4-butyl-2-hydroxycyclohepta-2,4,6-trienone: δH (DMSO, 500 MHz) 7.267-7.309 (1H, m), 7.142 (1H, s), 7.070-7.092 (1H, d, J=11), 6.910-6.931 (1H, d, J=10.5) 2.549-2.580 (2H, m), 1.503-1.564 (2H, m), 1.253-1.312 (2H, m), 0.860-0.889 (3H, m).

### Embodiment 7

### Synthesis of IST_009_025

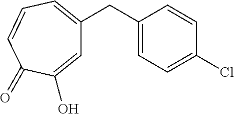 [SMILES: O=c1cccc(Cc2ccc(Cl)cc2)cc1O]

### Preparation of 4-(4-chlorobenzyl)-2-hydroxycyclohepta-2,4,6-trienone

Step 1:

At room temperature, to a 500 ml three-necked flask was added 150 ml of DMSO, stirred and purged with N₂ for 30 min to replace the air in the flask. Under the N₂ protection, potassium hydroxide (0.3 mol, 1 eq) was added and the mixture was continuously stirred for 0.5 h. Then cyclopentadiene monomers (0.8˜1.2 eq) were added dropwise within 30 min, and the mixture was continuously stirred for 1 h under the N₂ protection. Then, the mixture was cooled to about −10° C.; a mixed solution of n-hexane (150 ml) and p-chlorobenzyl bromide (0.8˜1.2 eq) was added dropwise into the three-necked flask for about 1 h, with the temperature controlled to be not higher than 20° C. And then the reaction continued for 1 h or more in a self-heating process; then, diluted hydrochloric acid was added dropwise to adjust the pH to be acidic and the mixture was fully stirred for 5-10 min and then separated; the water layer was extracted with 150 ml of n-hexane; the combined organic layers are washed with 250 ml of water twice until the water phase was neutral, dried over 40 g of anhydrous sodium sulfate and filtrated. The filtrate (about 400 ml) was added into a 1,000 ml three-necked flask and let standing for 7-20 h at a temperature of 20±2° C. to achieve isomerization wherein a GC was performed to determine the degree of isomerization, and p-chlorobenzyl bromide cyclopentadiene was obtained.

Step 2:

Under stirring, dichloroacetyl chloride (0.7˜1.5 eq) was added into the n-hexane solution of the p-chlorobenzyl bromide cyclopentadiene; after the mixture was cooled to about −15˜10° C., triethylamine (0.8˜1.5 eq) was added dropwise for about 1 h with the temperature controlled to be not higher than 15° C., and then the reaction proceeded for 1 h at a constant temperature; then, diluted hydrochloric acid was added dropwise to adjust the pH to be neutral or subacid; the solution was stirred for 5-10 min and then separated, wherein concentration of the product in each of the resulting organic phase and the water phase was determined by TLC (with PE as a developing solvent). The organic phase was then subjected to rotary evaporation at a temperature of 35° C. to remove solvent, and the residual liquid was dichlorobicycloketone.

Step 3:

The dichlorobicycloketone prepared in the previous step, 125 ml of tertiary butanol, 20 ml of acetic acid and 25 ml of water were added in sequence into a 1,000 ml three-necked flask with a condenser pipe and heated to a temperature of 75˜80° C. with stirring; 85 ml (98%, 0.675 mol) triethylamine was added dropwise within 1 hour, and then the resulting mixture was kept in a constant temperature overnight for reaction, and the reaction was monitored by TIC with PE as a developing solvent. At the completion of the reaction, the mixture was cooled to a temperature below 40° C. To the above reaction mixture were added with 100 ml of dichloromethane, 200 ml of water and 3 ml of concentrated hydrochloric acid and separated into a water phase and an organic layer. The organic layer was washed twice with 100 ml of water. The organic phase was added with 1M aqueous solution of potassium hydroxide for extraction. The water phase was washed twice with 100 ml of dichloromethane. Then the water phase was added with 100 ml of dichloromethane, and 2M diluted hydrochloric acid until it was acidic. The mixture was subject to extraction and separated. The organic phase was washed twice with distilled water and subjected to rotary evaporation to obtain a product, purity of which was 99% by HPLC. NMR result showed that the 4-(4-chlorobenzyl)-2-hydroxycyclohepta-2,4,6-trienone, a structural formula of which is:

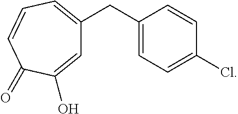 [SMILES: O=c1cccc(Cc2ccc(Cl)cc2)cc1O]

4-(4-chlorobenzyl)-2-hydroxycyclohepta-2,4,6-trienone: δH (DMSO, 500 MHz) 7.304-7.323 (2H, d, J=9.5), 7.213-7.284 (1H, m), 7.183 (1H, s), 7.157-7.139 (2H, d, J=9.5), 7.071-7.049 (1H, d, J=11), 6.986-7.006 (1H, d, J=10), 3.953 (2H, s).

### Embodiment 8

### Synthesis of IST_009_028

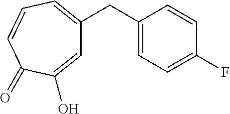 [SMILES: O=c1cccc(Cc2ccc(F)cc2)cc1O]

### Preparation of 4-(4-fluorobenzyl)-2-hydroxycyclohepta-2,4,6-trienone

Step 1:

At room temperature, to a 500 ml three-necked flask was added 150 ml DMSO is added, stirred and purged with N₂ for 30 min to replace the air in the flask. Under the N₂ protection, potassium hydroxide (0.3 mol, 1 eq) was added and the mixture was continuously stirred for 0.5 h. Then cyclopentadiene monomers (0.8˜1.2 eq) was added dropwise within 30 min, and the mixture was continuously stirred for 1 h under the N₂ protection. Then, the mixture was cooled to about −10° C. To the above mixture was added a mixed solution of n-hexane (150 ml) and p-fluorobenzyl bromide (0.8˜1.2 eq) within 1 h at the temperature controlled to be not higher than 20° C. And then the reaction continued for 1 h or more in a self-heating process; then, diluted hydrochloric acid was added dropwise to adjust the pH to be acidic and the mixture was fully stirred for 5-10 min and then separated; the water layer was extracted one more time with 150 ml of n-hexane; the combined organic layers were washed with 250 ml of water twice until the water phase was neutral, dried over 40 g of anhydrous sodium sulfate and filtrated. And then the filtrate (about 400 ml) is added into a 1,000 ml three-necked flask and let standing for 7-20 h at a temperature of 20±2° C. to achieve isomerization wherein a GC was performed to determine the degree of isomerization, and p-fluorobenzyl bromide cyclopentadiene was obtained.

Step 2:

Under stirring, dichloroacetyl chloride (0.7˜1.5 eq) was added into the n-hexane solution of the p-fluorobenzyl bromide cyclopentadiene; after the mixture was cooled to about −15˜10° C., triethylamine (0.8˜1.5 eq) was added dropwise within 1 h with the temperature controlled to be not higher than 15° C., and then the reaction proceeded for 1 h at a constant temperature; then, diluted hydrochloric acid was added dropwise to adjust the pH to be neutral or subacid; the resulting solution was stirred for 5-10 min and then separated, wherein concentration of the product in each of the resulting organic phase and the water phase was determined by TLC (with PE as a developing solvent). The organic phase was subjected to rotary evaporation at a temperature of 35° C. to remove solvent, and the residual liquid is dichlorobicycloketone.

Step 3:

The dichlorobicycloketone prepared in the previous step, 125 ml of tertiary butanol, 20 ml of acetic acid and 25 ml of water were added in sequence into a 1,000 ml three-necked flask with a condenser pipe and heated to a temperature of 75˜80° C. with stirring; 85 ml (98%, 0.675 mol) triethylamine was added dropwise within 1 hour, and then the mixture was kept in a constant temperature overnight, and the reaction was monitored by TLC with PE as a developing solvent. At the completion of the reaction, the resulting mixture was cooled to a temperature below 40° C. and added with 100 ml of dichloromethane, 200 ml of water and 3 ml of concentrated hydrochloric acid for extraction and thereafter separate into a water phase and an organic layer. The organic layer was washed twice with 100 ml of water. The organic phase was added with 1M aqueous solution of potassium hydroxide for extraction and separated. The water phase was washed twice with 100 ml of dichloromethane. Then the water phase was added with 100 ml of dichloromethane, and 2M diluted hydrochloric acid until it was acidic. The mixture was separated, and the organic phase was washed twice with distilled water and subjected to rotary evaporation to obtain a crude product.

The crude product was dissolved in about 50 ml of ethanol. The mixture was placed in a refrigerator overnight wherein solids was observed and collected by filtration. The filter cake was washed with about 5-10 ml of ethanol. The filtrate was subjected to rotary evaporation to obtain a product, the purity of which was 98% by HPLC. NMR result shows that the product was 4-(4-fluorobenzyl)-2-hydroxycyclohepta-2,4,6-trienone, a structural formula of which is:

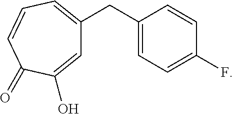 [SMILES: O=c1cccc(Cc2ccc(F)cc2)cc1O]

4-(4-fluorobenzyl)-2-hydroxycyclohepta-2,4,6-trienone: δH (DMSO, 500 MHz) 7.306-7.325 (2H, d, J=9.5), 7.183-7.285 (1H, m), 7.157 (1H, s), 7.120-7.139 (2H, d, J=9.5), 7.067-7.089 (1H, d, J=1), 6.973-6.993 (1H, d, J=10), 3.946 (2H, s).

### Embodiment 9

### Synthesis of IST_009_030 (2-hydroxy-4-n-octyl-2,4,6-cycloheptatriene-1-ketone)

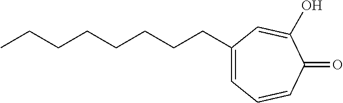 [SMILES: CCCCCCCCc1cccc(=O)c(O)c1]

### Preparation of 2-hydroxy-4-octylcyclohepta-2,4,6-trienone

Step 1:

At room temperature, to a 500 ml three-necked flask was added 150 ml DMSO is added, stirred and purged with N₂ for 30 min to replace the air in the flask. Under the N₂ protection, potassium hydroxide (0.3 mol, 1 eq) was added and the mixture was continuously stirred for 0.5 h. Then cyclopentadiene monomers (0.8˜1.2 eq) was added dropwise within 30 min, and the resulting mixture was continuously stirred for 1 h under the N₂ protection. Then, the mixture was cooled to about −10° C. To the above reaction mixture was added a mixed solution of n-hexane (150 ml) and 1-bromooctane (0.8˜1.2 eq) within 1 h, with the temperature controlled to be not higher than 20° C. And then the reaction continued for 1 h or more in a self-heating process; then, diluted hydrochloric acid was added dropwise to adjust the pH to be acidic; after the adjustment was completed, the mixture was fully stirred for 5-10 min and then separated; the water layer was extracted one more time with 150 ml of n-hexane; the combined organic layers were washed with 250 ml of water twice until the water phase was neutral, dried over 40 g of anhydrous sodium sulfate and filtrated, and then the filtrate (about 400 ml) is added into a 1,000 ml three-necked flask and let standing for 7-20 h at a temperature of 20±2° C. to achieve isomerization wherein a GC was performed to determine the degree of isomerization, and n-octyl cyclopentadiene was obtained.

Step 2:

Under stirring, dichloroacetyl chloride (0.7˜1.5 eq) was added into the n-hexane solution of the n-octyl cyclopentadiene; after the mixture was cooled to about −15˜10° C., triethylamine (0.8˜1.5 eq) was added dropwise for about 1 hr with the temperature controlled to be not higher than 15° C., and then the reaction proceeded for 1 h at a constant temperature; then, diluted hydrochloric acid was added dropwise to adjust the pH to be neutral or subacid; the solution was stirred for 5-10 min and then separated, wherein concentration of the product in each of the resulting organic phase and the water phase was determined by TLC (with PE as a developing solvent). The organic phase is then subjected to rotary evaporation at a temperature of 35° C. to remove solvent, and the residual green liquid is dichlorobicycloketone.

Step 3:

The dichlorobicycloketone prepared in the previous step, 125 ml of tertiary butanol, 20 ml of acetic acid and 25 ml of water were added in sequence into a 1,000 ml three-necked flask with a condenser pipe and heated to a temperature of 75˜80° C. with stirring; 85 ml (98%, 0.675 mol) triethylamine was added dropwise within 1 hour, and then the mixture was kept in a constant temperature overnight for reaction, and the reaction was monitored by TLC with PE as a developing solvent. At the completion of the reaction, the mixture was cooled to a temperature below 40° C. To the above reaction mixture was added 300 ml of dichloromethane resulting in precipitation of a great amount of solids. The solids were separated from the liquid, washed with petroleum ether, and then filtrated; the combined organic phases was added with about 200 ml of distilled water for extraction and thereafter separated. To the organic phase (the upper layer) was added 200 ml of water, and 150 ml of 3M aqueous solution of potassium hydroxide with stirring for extraction. The water layer was washed with petroleum ether. Then, into the water layer was added another 200 ml of petroleum ether and 3M diluted hydrochloric acid to adjust the pH value to 1-2 for extraction and thereafter separated. The organic layer was washed with water, and then separated with silica gel chromatography to obtain a product, the purity of which was 99% by HPLC. NMR result showed that the product was 2-hydroxy-4-octylcyclohepta-2,4,6-trienone, a structural formula of which is:

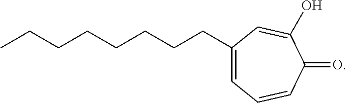 [SMILES: CCCCCCCCc1cccc(=O)c(O)c1]

2-hydroxy-4-octylcyclohepta-2,4,6-trienone: δH (DMSO, 500 MHz) 7.250-7.273 (1H, m), 7.133 (1H, s), 7.062-7.083 (1H, d, J=10.5), 6.097-6.927 (1H, d, J=10), 2.556-2.571 (2H, m), 1.530-1.544 (2H, m), 1.214-1.247 (10H, m), 0.814-0.841 (3H, m).

### Embodiment 10

### Synthesis of IST_009_031

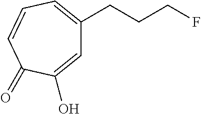 [SMILES: O=c1cccc(CCCF)cc1O]

### Preparation of 4-(3-fluoropropyl)-2-hydroxycyclohepta-2,4,6-trienone

Step 1:

At room temperature, to a 500 ml three-necked flask was added 150 ml DMSO is added, stirred and purged with N₂ for 30 min to replace the air in the flask. Under the N₂ protection, potassium hydroxide (0.3 mol, 1 eq) was added and the mixture was continuously stirred for 0.5 h. Then cyclopentadiene monomers (0.8˜1.2 eq) was added dropwise within 30 min, and the resulting mixture was continuously stirred for 1 h under the N₂ protection. Then, the mixture was cooled to about −10° C. and a mixed solution of n-hexane (150 ml) and 1-fluoro-3-bromopropane (0.8˜1.2 eq) was added dropwise into the three-necked flask for about 1 h, with the temperature controlled to be not higher than 20° C. And then the reaction continued for 1 h or more in a self-heating process; then, diluted hydrochloric acid was added dropwise to adjust the pH to be acidic and the mixture was fully stirred for 5-10 min and then separated; the water layer was extracted one more time with 150 ml of n-hexane; the combined organic layers were washed with 250 ml of water twice until the water phase was neutral, dried over 40 g of anhydrous sodium sulfate and filtrated. And then the filtrate (about 400 ml) was added into a 1,000 ml three-necked flask and let standing for 7-20 h at a temperature of 20±2° C. to achieve isomerization wherein a GC was performed to determine the degree of isomerization, and 3-fluoropropyl cyclopentadiene was obtained.

Step 2:

Under stirring, dichloroacetyl chloride (0.7˜1.5 eq) was added into the n-hexane solution of the 3-fluoropropyl cyclopentadiene; after the mixture was cooled to about −15˜10° C., triethylamine (0.8˜1.5 eq) was added dropwise for about 1 hr at the temperature controlled to be not higher than 15, and then the reaction proceeded for 1 h at a constant temperature; then, diluted hydrochloric acid was added dropwise to adjust the pH to be neutral or subacid; the solution was stirred for 5-10 min and then separated, wherein concentration of the product in each of the resulting organic phase and the water phase was determined by TLC (with PE as a developing solvent). The organic phase was then subjected to rotary evaporation at a temperature of 35° C. to remove solvent, and the residual liquid is dichlorobicycloketone.

Step 3:

The dichlorobicycloketone prepared in the previous step, 125 ml of tertiary butanol, 20 ml of acetic acid and 25 ml of water were added in sequence into a 1,000 ml three-necked flask with a condenser pipe and heated to a temperature of 75˜80° C. with stirring; 85 ml (98%, 0.675 mol) triethylamine was added dropwise (for about 1 h), and then the mixture was kept at a constant temperature overnight for reaction, and the reaction was monitored by TLC with PE as a developing solvent. At the completion of the reaction, the mixture was cooled to a temperature below 40° C. To the above reaction mixture were added 200 ml of ethyl acetate, 200 ml of water and 10 ml of concentrated hydrochloric acid for extraction and thereafter separated. The organic layer was washed twice with 200 ml of water and subjected to rotary evaporation to remove the solvent. The residue was subject to decompression distillation with a pressure of −0.1 MPa and a external temperature of 140˜150° C. The fraction at 130-135° C. was collected to give product as yellow liquid. NMR result showed that the product is 4-(3-fluoropropyl)-2-hydroxycyclohepta-2,4,6-trienone.

4-(3-fluoropropyl)-2-hydroxycyclohepta-2,4,6-trienone: δH (DMSO, 500 MHz) 7.277-7.319 (1H, m), 7.170 (1H, s), 7.083-7.105 (1H, d, J=11), 6.928-948 (1H, d, J=10), 4.484-4.508 (1H, m), 4.390-40401 (1H, m), 2.650-2.681 (2H, m), 1.892-1.997 (2H, m).

### Example 11

### Synthesis of IST_009_033

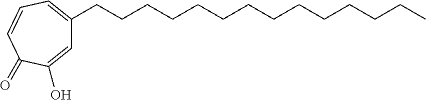 [SMILES: CCCCCCCCCCCCCCc1cccc(=O)c(O)c1]

### Preparation of 2-hydroxy-4-tetradecylcyclohepta-2,4,6-trienone

Step 1:

At room temperature, to a 500 ml three-necked flask was added 150 ml DMSO is added, stirred and purged with N₂ for 30 min to replace the air in the flask. Under the N₂ protection, potassium hydroxide (0.3 mol, 1 eq) was added and the mixture was continuously stirred for 0.5 h. Then cyclopentadiene monomers (0.8˜1.2 eq) was added dropwise within 30 min, and the resulting mixture was continuously stirred for 1 h under the N₂ protection. Then, the mixture was cooled to about −10° C. To the above mixture was added a mixed solution of n-hexane (150 ml) and bromotetradecane (0.8˜1.2 eq) dropwise within 1 h at the temperature controlled to be not higher than 20° C. And then the reaction continued for 1 h or more in a self-heating process; then, diluted hydrochloric acid was added dropwise to adjust the pH to be acidic and the mixture was fully stirred for 5-10 min and then separated; the water layer was extracted one more time with 150 ml of n-hexane; the combined organic layers were washed with 250 ml of water twice until the water phase was neutral, dried over 40 g of anhydrous sodium sulfate and filtrated. And then the filtrate (about 400 ml) was added into a 1,000 ml three-necked flask and let standing for 7-20 h at a temperature of 20±2° C. to achieve isomerization wherein a GC was performed to determine the degree of isomerization, and n-tetradecyl cyclopentadiene was obtained.

Step 2:

Under stirring, dichloroacetyl chloride (0.7˜1.5 eq) was added into the n-hexane solution of the n-tetradecyl cyclopentadiene; after the mixture was cooled to about −15˜10° C., triethylamine (0.8˜1.5 eq) was added dropwise for about 1 hr at the temperature controlled to be not higher than 15° C., and then the reaction proceeded for 1 h at a constant temperature; then, diluted hydrochloric acid was added dropwise to adjust the pH to be neutral or subacid; the solution was stirred for 5-10 min and then separated, wherein concentration of the product in each of the resulting organic phase and the water phase was determined by TLC (with PE as a developing solvent). The organic phase was then subjected to rotary evaporation at a temperature of 35° C. to remove solvent, and the residual liquid was dichlorobicycloketone.

Step 3:

The dichlorobicycloketone prepared in the previous step, 125 ml of tertiary butanol, 20 ml of acetic acid and 25 ml of water were added in sequence into a 1,000 ml three-necked flask with a condenser pipe and heated to a temperature of 75˜80° C. with stirring; 85 ml (98%, 0.675 mol) triethylamine was added dropwise (for about 1 h), and then the mixture was kept in a constant temperature overnight for reaction, and the reaction was monitored by TLC with PE as a developing solvent. At the completion of the reaction, the mixture was cooled to a temperature below 40° C. To the resulting mixture were added 100 ml of petroleum ether and 100 ml of water for extraction and thereafter separated. The organic phase was mixed with silica gel and then subjected to column chromatography (PE/EA=10:1) to give the product. NMR result showed that the product was 2-hydroxy-4-tetradecylcyclohepta-2,4,6-trienone, a structural formula of which is:

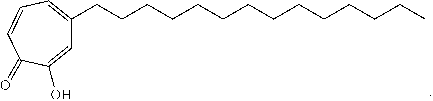 [SMILES: CCCCCCCCCCCCCCc1cccc(=O)c(O)c1]

2-hydroxy-4-tetradecylcyclohepta-2,4,6-trienone: δH (DMSO, 500 MHz) 7.273-7.340 (1H, m), 7.145 (1H, s), 7.073-7.095 (1H, d, J=11), 6.920-6.940 (1H, d, J=10), 2.506-2.587 (2H, m), 1.561 (2H, m), 1.227-1.275 (22H, m), 0.835-0.861 (3H, m).

### Example 12

### In Vitro Antibacterial Activity (MICs) of β-Hinokitiol Derivatives

Doubling dilutions were performed to determine the minimum inhibitory concentration of β-hinokitiol derivatives against different bacteria in vitro. Tables I to 4 were results of the test. Tables I to 4 showed that the β-hinokitiol derivatives had broad-spectrum antibacterial activities and showed high inhibitory activities of on major animal pathogenic bacteria (for example, clostridium perfringens, staphylococcus aureus, escherichia coli, salmonella, riemerella anatipestifer, haemophilus parasuis, vibrio and streptococcus), and in particular that, the antibacterial activities of additives such as IST_009_001 and IST_009_003 were better than those of the lead compound IST_001_002 (β-hinokitiol).

TABLE 1
Minimum inhibitory concentration of β-hinokitiol
derivatives against clostridium perfringens and
staphylococcus aureus in vitro (MIC, μg/ml)

| | Bacterial strain | | | |
|---|---|---|---|---|
| | | | Staphylo- | Staphylo- |
| | | | coccus | coccus |
| | Clostridium | Clostridium | aureus | aureus |
| Compound | JM1210B | JM1212A | SA10 | PNB14 |
| IST_009_003 | 6.25 | 6.25 | ND | ND |
| IST_009_008 | 12.5 | 12.5 | ND | ND |
| IST_009_033 | 50 | 50 | ND | ND |
| IST_001_002 | 6.25 | 12.5 | 25 | 25 |
| IST_009_001 | 3.13 | 3.13 | 12.5 | 12.5 |
| IST_009_004 | 12.5 | 12.5 | ND | ND |
| IST_009_018 | 3.13 | 3.13 | ND | ND |
| IST_009_019 | 12.5 | 6.25 | ND | ND |
| IST_009_030 | 25 | 12.5 | 12.5 | 12.5 |
| IST_009_031 | 6.25 | 6.25 | ND | ND |

Note:
ND represents “Not Done”.

TABLE 2
Minimum inhibitory concentration of β-hinokitiol derivatives against
escherichia coli and salmonellas in vitro (MIC, μg/ml)

| | Bacterial strain | | | |
|---|---|---|---|---|
| | Salmo- | Salmo- | Eserichia | Eserichia |
| | nellas | nellas | coli | coli |
| Compound | FS1303B | GD1303-3 | SS1107A | HZ1308-1 |
| IST_009_003 | 6.25 | 12.5 | 12.5 | 6.25 |
| IST_009_008 | 25 | 25 | 25 | 25 |
| IST_009_033 | >100 | >100 | >100 | >100 |
| IST_001_002 | 25 | 25 | 25 | 25 |
| IST_009_001 | 6.25 | 12.5 | 12.5 | 12.5 |
| IST_009_004 | 25 | 25 | 25 | 12.5 |
| IST_009_018 | 12.5 | 25 | 50 | 25 |
| IST_009_019 | 25 | 25 | 25 | 25 |
| IST_009_030 | 25 | >100 | >100 | 25 |
| IST_009_031 | 50 | 50 | 100 | 25 |

TABLE 3
Minimum inhibitory concentration of β-hinokitiol
derivatives against haemophilus parasuis and riemerella anatipestifer
in vitro (MIC, μg/ml)

| | Bacterial strain | | | |
|---|---|---|---|---|
| | Haemophilus | Haemophilus | Riemerella | Riemerella |
| | parasuis | parasuis | anatipestifer | anatipestifer |
| Compound | 2-3A | S5A | WS | SCAU03 |
| IST_009_003 | 0.2 | 0.2 | 3.13 | 12.5 |
| IST_009_008 | 0.39 | 0.39 | 6.25 | 12.5 |
| IST_009_033 | 50 | 50 | 25 | 100 |
| IST_001_002 | 0.78 | 0.78 | 6.25 | 25 |
| IST_009_001 | 0.39 | 0.2 | 3.13 | 12.5 |
| IST_009_004 | 0.78 | 0.2 | 6.25 | 12.5 |
| IST_009_018 | 3.13 | 1.56 | 6.25 | 12.5 |
| IST_009_019 | 0.39 | 0.2 | ND | ND |
| IST_009_030 | 6.25 | 0.39 | 3.13 | 12.5 |
| IST_009_031 | 6.25 | 3.13 | ND | ND |

Note:
ND represents “Not Done”.

TABLE 4
Minimum inhibitory concentration of β-hinokitiol derivatives against
vibrio and streptococcus In vitro (MIC, μg/ml)

| | Bacterial strain | | | |
|---|---|---|---|---|
| | Vibrio | Vibrio | Streptococcus | Streptococcus |
| Compound | VP3 | VP5 | TS1205-2 | KP1307-1 |
| IST_001_002 | 25 | 25 | 50 | 50 |
| IST_009_003 | 50 | 25 | 12.5 | 12.5 |
| IST_009_008 | 50 | 25 | 50 | 50 |
| IST_009_033 | >100 | >100 | >100 | >100 |
| IST_009_001 | 12.5 | 6.25 | 25 | 25 |
| IST_009_004 | 50 | 50 | 50 | 6.25 |
| IST_009_018 | 25 | 25 | >100 | 12.5 |
| IST_009_019 | 50 | 50 | 25 | >100 |
| IST_009_025 | >100 | >100 | 25 | 25 |
| IST_009_028 | 100 | 100 | 25 | 25 |
| IST_009_030 | >100 | >100 | 25 | 25 |

### Example 13

### Application Effects of β-hinokitiol Derivatives in Broiler Feeds

598 fast-grown type yellow feather broilers (female) aged 30-days were divided into five groups according to table 5. Each group was respectively added with β-hinokitiol derivatives or/and colistin sulfate. During the test, the broilers ate food and drank water freely. The test lasted for 14 days. Results showed that β-hinokitiol derivatives such as IST_009_001 and IST_009_003 could enhance the weight gain and the feed conversion efficiency of the experimental broilers (table 6).

TABLE 5
Grouping of application effect test of β-hinokitiol
derivatives in broiler feeds

| | Quantity of the | | Dosage | |
|---|---|---|---|---|
| Group | broilers | Additives | (ppm) | Administration |
| 1 | 39 | — | — | Mixed with feed |
| 2 | 40 | IST_009_001 | 5 | Mixed with feed |
| 3 | 40 | IST_009_001 | 15 | Mixed with feed |
| 4 | 40 | IST_009_003 | 40 | Mixed with feed |
| 5 | 40 | IST_009_001 + | 10 + 20 | Mixed with feed |
| | | colistin sulfate | | |
| 6 | 40 | — | — | Mixed with feed |
| 7 | 40 | IST_009_001 | 5 | Mixed with feed |
| 8 | 40 | IST_009_001 | 15 | Mixed with feed |
| 9 | 40 | IST_009_003 | 40 | Mixed with feed |
| 10 | 40 | IST_009_001 + | 10 + 20 | Mixed with feed |
| | | colistin sulfate | | |
| 11 | 40 | — | — | Mixed with feed |
| 12 | 40 | IST_009_001 | 5 | Mixed with feed |
| 13 | 40 | IST_009_001 | 15 | Mixed with feed |
| 14 | 39 | IST_009_003 | 40 | Mixed with feed |
| 15 | 40 | IST_009_001 + | 10 + 20 | Mixed with feed |
| | | colistin sulfate | | |

TABLE 6
Results of application effect test of β-hinokitiol derivatives in broiler feeds

| | | | | Total | Average daily | feed |
|---|---|---|---|---|---|---|
| | | Dosage | Average weight | consumption | weight gain | conversion |
| Group | Additives | (ppm) | gain (g) | (kg) | (g/d * bird) | efficiency |
| 1 | — | — | 475.16 | 54.67 | 32.01 | 3.049 |
| 2 | IST_009_001 | 5 | 547.21 | 55.12 | 33.52 | 2.808 |
| 3 | IST_009_001 | 15 | 523.38 | 57.84 | 34.53 | 2.889 |
| 4 | IST_009_003 | 40 | 502.05 | 58.05 | 36.21 | 2.862 |
| 5 | 1ST 009 001 + colistin | 10 + 20 | 528.50 | 57.63 | 36.82 | 2.795 |
| | sulfate | | | | | |
| 6 | — | — | 517.00 | 60.27 | 36.67 | 2.812 |
| 7 | IST_009_001 | 5 | 516.79 | 61 | 37.75 | 2.886 |
| 8 | IST_009_001 | 15 | 517.17 | 58.03 | 36.94 | 2.807 |
| 9 | IST_009_003 | 40 | 449.90 | 57.71 | 34.19 | 2.952 |
| 10 | 1ST 009 001 + colistin | 10 + 20 | 507.00 | 60.39 | 38.18 | 2.825 |
| | sulfate | | | | | |
| 11 | — | — | 507.24 | 60.55 | 35.13 | 2.980 |
| 12 | IST_009_001 | 5 | 520.00 | 58.85 | 36.93 | 2.846 |
| 13 | IST_009_001 | 15 | 525.50 | 57.61 | 37.5 | 2.741 |
| 14 | IST_009_003 | 40 | 534.50 | 58.17 | 37.14 | 2.868 |
| 15 | IST_009_001 + colistin | 10 + 20 | 527.00 | 56.61 | 37.64 | 2.685 |
| | sulfate | | | | | |

### Example 14

### Effect of β-Hinokitiol Derivatives on Diarrhea of Weaned Pigs

80 25-day-old weaned pigs were randomly divided into eight groups according to table 7, 10 pigs in each group. The basic diets without any antibiotics were continuously added with IST_009_001, colistin sulfate or IST_001_002 according to the dosage in the table. During the testing, the pigs ate food and drank water freely with temperature preservation. The test lasted for 10 days. Results (table 7) showed that, adding IST_009_001 in the feeds could obviously reduce the diarrhea rate of the tested pigs, and the effect of the IST_009_001 30 ppm test group was obviously better than that of colistin sulfate or IST_001_002 groups.

TABLE 7
Effect of β-hinokitiol derivatives on diarrhea of weaned pigs

| | | Dosage | Average daily |
|---|---|---|---|
| Group | Additives | (ppm) | diarrhea rate (%) |
| 1 | Blank group | 0 | 52 |
| 2 | Blank group | 0 | 57 |
| 3 | Colistin sulfate | 60 | 30 |
| 4 | Colistin sulfate | 60 | 33 |
| 5 | IST_009_001 | 30 | 16 |
| 6 | IST_009_001 | 30 | 17 |
| 7 | IST_001_002 | 30 | 35 |
| 8 | IST_001_002 | 30 | 32 |

### Embodiment 15

### Protection Effect of β-Hinokitiol Derivatives Against Attacking and Infection of riemerella anatipestifer

100 16-day-old cherry valley ducks were randomly divided into 10 test groups according to table 8. Each duck was injected with riemerella anatipestifer of 5×10⁷ CFU via intramuscular injection for the purpose of attacking and infection, and injected with the medicines via intramuscular injection simultaneously. Morbidity and death of the tested ducks were observed, and dead ducks were subject to autopsy to verify whether the death was caused by riemerella anatipestifer infection. The observation lasted for 7 days. All tested ducks were executed at the end of the test. Autopsy were performed to confirm the morbidity. The statistic results can be seen in table 8. Table 8 showed that the β-hinokitiol derivative IST_009_001 could protect ducks against attacking and infection of riemerella anatipestifer.

TABLE 8
Protection effect of β-hinokitiol derivatives against attacking
and infection of riemerella anatipestifer

| | | Dosage of | | | | |
|---|---|---|---|---|---|---|
| | | riemerella | | | | |
| | Quantity of | anatipestifer | | Dosage | Morbidity | Death |
| Group | the ducks | (CFU/bird) | Medicine | (mg/kg weight) | (%) | rate (%) |
| 1 | 20 | — | — | — | 0 | 0 |
| 2 | 20 | 5 × 10⁷ | — | — | 100 | 65 |
| 3 | 20 | 5 × 10⁷ | Cefotaxime | 10 | 25 | 0 |
| 4 | 20 | 5 × 10⁷ | IST_009_001 | 10 | 40 | 0 |
| 5 | 20 | 5 × 10⁷ | IST_009_001 | 20 | 25 | 0 |

1. β-hinokitiol derivatives, having a structural formula as shown in formula (I): 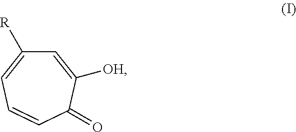
wherein, R is n-propyl, n-pentyl, benzyl, sec-butyl, ethyl, n-butyl, p-chlorobenzyl, p-fluorobenzyl, n-octyl, 1-fluoropropyl or n-tetradecyl.

2. A method of using any one of the β-hinokitiol derivatives according to claim 1 as an animal antibacterial agent, comprising the step of administering the animal antibacterial agent to an animal.

3. The method according to claim 2, wherein said animal includes pigs, chickens, ducks, geese, beef cattle, dairy cattle, sheep, fish, shrimp, foxes, martens or raccoon dogs in all growth stages.

4. (canceled)

5. The method according to claim 2, wherein, when the animal antibacterial agent used to treat diseases caused by infection with various animals sensitive bacteria, the dosage is 1-30 mg/kg body weight.

6. A method of using any one of the β-hinokitiol derivatives according to claim 1 in preparation of antibacterial growth promoters comprising the step of adding the any one of the β-hinokitiol derivatives to animal feed.

7. The method according to claim 6, wherein said animal includes pigs, chickens, ducks, geese, beef cattle, dairy cattle, sheep, fish, shrimp, foxes, martens or raccoon dogs in all growth stages.

8. The method according to claim 6, wherein an additive dosage of said β-hinokitiol derivative in animal feed is 0.1˜200 ppm.
